SEQUENCE LISTING (5 sequences)

SEQ ID NO: 1 - DNA, 1745 bp, tobacco plant
tctagatcgtactaccaccactacgccgccatggttctgtccaagacagcgtcggaaagtgacgtctccatccactccactttcgcttcccgatatgttcgaacttctcttcccaggtttaagatgccagagaattcaataccaaaggaagcagcatatcagattataaatgatgagcttatgttagatggaaatccaaggctaaatttagcatctttcgttacaacatggatggagccagaatgtaatacgttaatgatggattccattaacaagaactacgttgacatggatgaataccctgtaaccactgagcttcagaatcgatgtgtaaatatgatagctcatttgtttaatgcaccacttggagatggagagactgcagttggagttggaactgttggatcctctgaagctattatgcttgctggattagcctttaaaagaaaatggcaaaataaaatgaaagcccaaggcaagccctttgataagcccaatatcgtcaccggtgctaatgtccaggtgtgttgggagaaatttgcaaggtattttgaagtggagttgaaagaagtaaaattgagtgatggatactatgtgatggaccctgagaaagctgtggaaatggtggatgagaataccatttgtgttgctgctatcttaggttcaacactcaatggtgaatttgaagatgttaagcgtttgaatgaccttttgattgagaagaacaaagaaaccgggtgggacactccaattcatgtggatgcagcaagtggtggatttattgcaccattcctttatccagagcttgaatgggactttagattgccattggtgaagagtattaatgtgagtggtcacaaatatggtcttgtctatgctggtattggttgggccatttggaggaataaggaagacttgcctgatgaacttattttccacatcaattaccttggtgctgatcaacctactttcactctcaacttctctaaaggttctagccaagtaattgctcaatattaccaacttattcgcttgggttttgagggttacaagaatgttatggagaattgtcaagaaaatgcaagggtattaagagaaggaattgaaaaaagtggaagattcaacataatctccaaagaaattggagttcccttagtagcattttctcttaaagacaacagtcaacacaatgagttcgaaatttctgaaactcttagaagatttggatggattgttcctgcatatactatgccaccaaatgctcaacatgttacagttctcagagttgtcattagagaagatttctcccgcacactagcggagcgactggtaatagacattgaaaaagtcctccacgagctagacacacttccggcgagggtcaacgctaagctagccgtggccgaggcgaatggcagcggcgtgcataagaaaacagatagagaagtgcagctagagattactactgcatggaagaaatttgttgctgataagaagaagaagactaatggagtttgttaatttaatttaacaaaaaaaaagtttataatatggtgatttatgtaactactagcagtcgtactgcttgttttttatatttgagttgatgtgttttttgagcacttgaggagctagctagttattgctagtgaaaaattggatgatatattttggactactttgtaagtttgtattattaatccaaattaaacgatatttatcatgcaaaaaaaaaaaaaaaaaaa

SEQ ID NO: 2 - protein, 496 aa, tobacco plant
MVLSKTASESDVSIHSTFASRYVRTSLPRFKMPENSIPKEAAYQIINDELMLDGNPRLNLASFVTTWMEPECNTLMMDSINKNYVDMDEYPVTTELQNRCVNMIAHLFNAPLGDGETAVGVGTVGSSEAIMLAGLAFKRKWQNKMKAQGKPFDKPNIVTGANVQVCWEKFARYFEVELKEVKLSDGYYVMDPEKAVEMVDENTICVAAILGSTLNGEFEDVKRLNDLLIEKNKETGWDTPIHVDAASGGFIAPFLYPELEWDFRLPLVKSINVSGHKYGLVYAGIGWAIWRNKEDLPDELIFHINYLGADQPTFTLNFSKGSSQVIAQYYQLIRLGFEGYKNVMENCQENARVLREGIEKSGRFNIISKEIGVPLVAFSLKDNSQHNEFEISETLRRFGWIVPAYTMPPNAGHVTVLRVVIREDFSRTLAERLVIDIEKVLHELDTLPARVNAKLAVAEANGSGVHKKTDREVQLEITTAWKKFVADKKKKTNGVC

SEQ ID NO: 3 - DNA, 21 bp, Artificial Sequence
  PCR primer
ggagtccatcataagcttatt

SEQ ID NO: 4 - DNA, 29 bp, Artificial Sequence
  PCR primer
cttctagatcgtactaccaccactacgcc

SEQ ID NO: 5 - DNA, 34 bp, Artificial Sequence
  PCR primer
gaagttcctatactttctagagaataggaacttc

(19) United States
(12) Patent Application Publication
(10) Pub. No.: US 2003/0110530 A1
(43) Pub. Date: Jun. 12, 2003

(54) Transgenic plants having reduced susceptibility to invertebrate pests

(76) Inventors: Barry Shelp, Guelph (CA); Alan Bown, Saint Catherines (CA)

Correspondence Address: OGILVY RENAULT, 1981 MCGILL COLLEGE AVENUE, SUITE 1600, MONTREAL, QC, H3A2Y3, CA

(21) Appl. No.: 10/005,602

(22) Filed: Dec. 7, 2001

(51) Int. Cl. A01H 5/00; C12N 15/82

(52) U.S. Cl. 800/279

(57) ABSTRACT

A novel transgenic plant having enhanced resistance to invertebrate pests is provided. The plant is transformed with a recombinant nucleic acid encoding a functional glutamate decarboxylase (GAD). A method of producing the transgenic plant is also provided.

### FIELD OF THE INVENTION

The present invention relates to transgenic plants which have reduced susceptibility to invertebrate pests. In particular, the present invention relates to transgenic plants that overexpress glutamate decarboxylase, and to methods for preparing such transgenic plants.

### BACKGROUND OF THE INVENTION

Parasitic nematodes infect a wide range of important field, vegetable, fruit and ornamental plants, and are responsible for 10-12% yield losses on average (Barker et al. 1994 J Nematol. 26:127-137; Sasser and Freckman 1987 In Veech & Dickson (eds), Vistas on Nematology, Society Nematologists Inc, Hyattsville, Mo. Pp 7-14). There are over 50 genera of plant-parasitic nematodes (Wyss 1997 In Fenoll et al (eds), Cellular and Molecular Aspects of Plant-Nematode Interactions, Kluwer Acad Publ., Dordrecht, Netherlands. Pp 5-22). The females of root nematodes, which have the highest economic impact, exhibit a prolonged sedentary phase during which they modify plant cells into feeding sites. Root knot nematodes (Meloidogyne spp.) are responsible for the majority of the annual worldwide losses of C$150 billion attributed to nematode damage (Meyer 1999 http://www.primenet.com/˜scottm/nl.html). They are particularly active in warmer soils, and highly unusual in that they are able to attack a very wide range of hosts. The tobacco crop in the USA alone (farm gate value of CS4 billion) faces annual yield losses of about C$200 million due to the root-knot nematode. Cyst nematodes (Heterodera and Globodera spp.) are a second group of root-sedentary nematodes that are the predominant pests of temperate agriculture. Each species has a narrow host range and the species that attack potato, sugar beet and soybean are of particular importance. The soybean cyst nematode (Heterodera glycines) is responsible for approximately C$400 million of annual damage in the USA, and C$10 million of annual damage in Ontario to the soybean crop, which has a total farm gate value of about C$16 billion in North America (Meyer 1999; Plowright & Bridge 1990 Nematol 36:81; Tenuta et al. February 1999 SCN Handbook in Country Guide Magazine).

Chemicals, cultural practices and resistant cultivars are currently used to control nematodes, and are often used in an integrated manner. In Ontario, Canada, the tobacco and strawberry industries (farm gate values of C$250 M and C$19 M, respectively) are totally dependent on soil fumigation to control nematodes (Marcotte & Tibelius 1998 Improving Food and Agriculture Productivity and the Environment: Canadian Initiatives in Methyl Bromide Alternatives and Emission Control Technologies, Environment Canada. Pp 46). In 1993, in excess of 1.5 M L of the Telon- and Vorlex-brand formulations were applied to tobacco at a cost of about $200 per acre. In field crops such as soybean (farm gate value of C$500 M), the problem is more difficult in some ways, because fumigation is economically prohibitive (i.e., soybean crop is valued at only $300 per acre). The active ingredients of fumigant formulations are either known carcinogens or chlorinated hydrocarbons (which contribute to the deterioration of the environment, with a particular impact on the ozone layer). In Canada and the United States, legislation has already started to restrict or to eliminate the usage of these ingredients (Nolling & Becker 1994 J Nematol 26:5761; Marcotte & Tilbelius 1998). Other chemical treatments involving synthetic nematicidal compounds are only available for a limited number of crops and present opportunities for the development of chemical resistance (Casida 1993 Arch Insect Biochem Physiol 22:13). These primarily include nerve poisons which are hazardous to human health. Resistant cultivars have been a commercial success for a limited range of crops, but this breeding approach is time consuming and unable to control many nematode problems for a variety of reasons (e.g. temperature sensitivity, species or pathotype-specificity) Niebel et al. 1994 Parasitol Today 10:424, Bridge 1996 Annu Rev Phytopathol 34:201). The nematode-resistant soybean cultivars available in Ontario produce as well as susceptible cultivars on non-infested soils, and 30-50% more on infested soils (Ontario Oil and Protein Seed Crop Committee, 2001).

Insects also attack a variety of important crop plants. Total worldwide losses of the eight principal food and cash crops (coffee, potato, soybean, maize, barley, cotton, rice and wheat) due to insect damage, are estimated at 15.6% of total production (C$130 billion) (Duck & Evola in Advances In Insect Control: The Role of Transgenic Plants, 1997, Carozzi & Koziel (eds.), Taylor & Francis Ltd., Bristol, Pa., pp 1-20) In North American alone, the losses are estimated at bout $C7 billion Of particular interest are insect species that feed on roots. Examples include European corn borers (Ostrinia nubililas), corn rootworm (Drabrotica sp.) and wireworm (Limonius spp.).

The management of insect pests also currently requires the use of chemical treatments that present human health and environmental hazards (Advances In Insect Control: The Role of Transgenic Plants, 1997, Carozzi & Koziel (eds.), Taylor & Francis Ltd, Bristol, Pa. Pp 301). Thus profitable, safe and sustainable biological alternatives to chemical pesticides are needed for the management of nematode and insect pests. The limitations of conventional control measures provide an excellent opportunity for plant genetic engineering to produce novel and effective forms of control. Genetic engineering can provide the means to rapidly introduce a pest resistance gene into locally-adapted cultivars, thereby improving yields in areas infested with invertebrate pests, and providing breeding material for the production of cultivars suitable for specific environments.

In 1997, corn, potato and cotton plants expressing a Bacillus thuringiensis endotoxin gene were first introduced in the marketplace. Some evidence suggests that the pests being targeted are capable of developing resistance to this product although the problem is to some extent overcome by the use of refugia (Advances In Insect Control: The Role of Transgenic Plants, 1997, Carozzi & Koziel (eds.), Taylor & Francis Ltd., Bristol Pa. Pp 301). In order to minimize the chances that nematode and insect pests evolve mechanisms to overcome or circumvent the plant's resistance, it is important to develop new resistance strategies targeted at reducing the number of pest offspring, rather than complete removal of the pest, and to produce commercial cultivars with more than one resistance mechanism (i.e. gene pyramiding).

Several current pesticides function by interfering with the 4-aminobutyrate (GABA)-gated chloride channel in the central nervous system of insects and nematodes (Casida, 1993 Arch.Insect Biochem. Physol. 22:13). GABA is a naturally-occurring inhibitory neurotransmitter which has ready access to the nervous system of invertebrates, but not that of vertebrates such as man, and has been shown to deter insect grow and development (Ramputh and Bown, 1996 Plant Physio. 111:1349). Typically, GABA levels are low in plants (ranging from 0.03 to 2.00 μmol/g fresh weight (FW)), but increase several fold in response to many diverse stimuli such as insect walking and feeding (i.e. biotic stress) and temperature shock (i.e. abiotic stress). This result a be attributed to increases in cytosolic H+ or calcium/calmodulin levels which directly affect the activity of the enzyme responsible for the synthesis of GABA, namely glutamate decarboxylase (GAD) (FIG. 1; Shelp et al., 1999 Tr. Plant Sci. 4:446). In particular, calcium/calmodulin binds to a carboxyl-terminal domain on the GAD gene, thereby relieving the autoinhibition of GAD activity. Although the endogenous synthesis of GABA appears to serve as a plant defense mechanism, further increases in GABA levels may lead to corresponding reductions in damage due to invertebrate pests Transgenic plants which overexpress GAD and thereby cause GABA accumulation have been prepared; however, these plants do not express invertebrate pest-resistant root GABA levels and have questionable utility in that they exhibit severe morphological abnormalities (Baum et al., 1996 EMBO Journal, 15:2988).

Therefore, it would be desirable to develop phenotypically normal plants having in creased GABA levels in order to reduce damage by invertebrate pests.

### SUMMARY OF THE INVENTION

Accordingly, in one aspect, the present invention provides a phenotypically normal transgenic plant having reduced susceptibility to invertebrate pests, wherein the plant is transformed with a nucleic acid encoding glutamate decarboxylase.

In a further aspect of the present invention, a method of producing a phenotypically normal transgenic plant with reduced susceptibility to invertebrate pests, comprising:

1) transforming a recipient plant cell with a recombinant nucleic acid encoding functional glutamate decarboxylase;

2) generating a plant from the transformed plant cell; and

3) selecting for a phenotypically normal transformed plant having a GABA level of at least 100 nmol/g FW (fresh weight).

These and other aspects of the present invention will be described by reference to the following figures in which:

### BRIEF DESCRIPTION OF THE FIGURES

FIG. 1 is the nucleic acid (SEQ ID No: 1) and amino acid (SEQ ID No: 2) sequence of tobacco glutamate decarboxylase (GAD);

FIG. 2 illustrates the DNA construct used to prepare the transgenic plants of the present invention; and

FIG. 3 graphically shows the percentage increase in GABA accumulation during stress, as well as the susceptibility to the root knot nematode in 12 week-old tobacco plants.

### DETAILED DESCRIPTION OF THE INVENTION

A phenotypically normal transgenic plant is provided having reduced susceptibility to invertebrate pests. The plant is transformed with a nucleic acid encoding glutamate decarboxylase and expresses GABA levels of at least 100 nmol/g fresh weight FW).

The term “phenotypically normal” as it is used herein with respect to the present transgenic plants refers to the fact that the physical characteristics of the transgenic plants, such as height, leaf size and colour, root size and the properties of the flower including ability to reproduce, appear substantially similar to the same characteristics of a corresponding wild-type plant, and retain substantial function.

The term “reduced susceptibility” is used herein to denote the resistance of the present transgenic plants to invertebrate pests. A plant is said to be less susceptible to plant parasitic nematodes if a statistically significant decrease in the number of maturing (i.e. reproductive) female parasitic nematodes developing at the surface of plant roots can be observed as compared to control plants. Reduced susceptibility/resistance classification according to the number of mature females is standard practice for cyst- and root-knot nematodes (e.g. LaMontia 1991, Plant Disease 75: 453 -454; Omwega et al. 1990 Phytopathology 80: 745-748). Alteratively, a plant may be said to be less susceptible to plant parasitic nematodes if its use significantly decreases nematode population density in the soil and roots after a period of time, and protection is extended to the following susceptible crop ( Johnson 1998 In Plant and Nematode Interactions 1998 Agronomy Monograph No. 36, Amer. Soc. Agronomy, Crop Science Soc. Amer., Soil Science Soc. Amer., Madison, Wis., pp. 595-635). Within the context of the invention, a plant is said to be less susceptible to an insect pest if significantly less time is spent by an insect walking on, feeding on or living on the plant, or if significantly less of the vegetative (i.e. foliage, stems, roots) or reproductive tissues (i.e. flowers, seeds) of a plant is eaten or damaged by an insect (Rausher 1992 In Rottberg and Isman (eds) Insect Chemical Ecology, Chapman & Hall Publ, Pp 42). Alternatively, a plant is said to be less susceptible to an insect pest if its use decreases the rate of development, maturation or population density of an insect near the plant after a period of time (Ramputh and Down, supra).

According to the present invention, transgenic plants having an enhanced capacity to accumulate GABA is achieved by sense expression of homologous or heterologous GAD genes. The term “homologous” refers to genes obtainable from the same plant species as the plant host, while the term “heterologous” refers to genes from a different plant or non-plant species. Heterologous genes also comprise synthetic analogs of genes which diverge in the mRNA encoding nucleic ad sequence by at least 5% of the host gene's sequence. As genes are often highly conserved, heterologous probes from other (plant) species can be used to isolate the corresponding gene from the crop species that is to be made resistant For plants in particular, GAD genes have been isolated from Petunia (awn et al 1993 J Biol. Chem. 268: 19610-19617), tomato (Gallego et al. 1995 Plant Mol. Biol. 27: 1143-1151), tobacco (Yu & Oh 1998 Mol. Cell 8: 125-129, Yevtushenko et al. 1999 Abstr. Annu. Meeting of the Amer. Soc. Plant Physiol., Baltimore, #375) and Arabidopsis (Turano & Fang 1998 Plant Physiol. 117: 1411-1421; Zik et al.1998 Plant Mol. Biol. 37: 967-975). Up to five GAD isoforms have been identified in Arabidopsis (Shelp et al. 1999 Tr. Plant Sci 4: 446-452).

GAD-encoding nucleic acid can be prepared by applying selected techniques of gene isolation or gene synthesis as a first step. As described in more detail in the examples herein, GAD polynucleotides can be obtained by careful application of conventional gene isolation and cloning techniques. This typically will entail extraction of total messenger RNA from a fresh source of plant tissue, followed by conversion of message to cDNA and formation of a cDNA library in plasmidic vectors. The cDNA library is then probed using a labeled nucleic acid fragment derived from a gene believed to be highly homologous to the cDNA of interest. Hybridizing cDNA clones are further screened and positive clones are prepared for insertion into an expression vector.

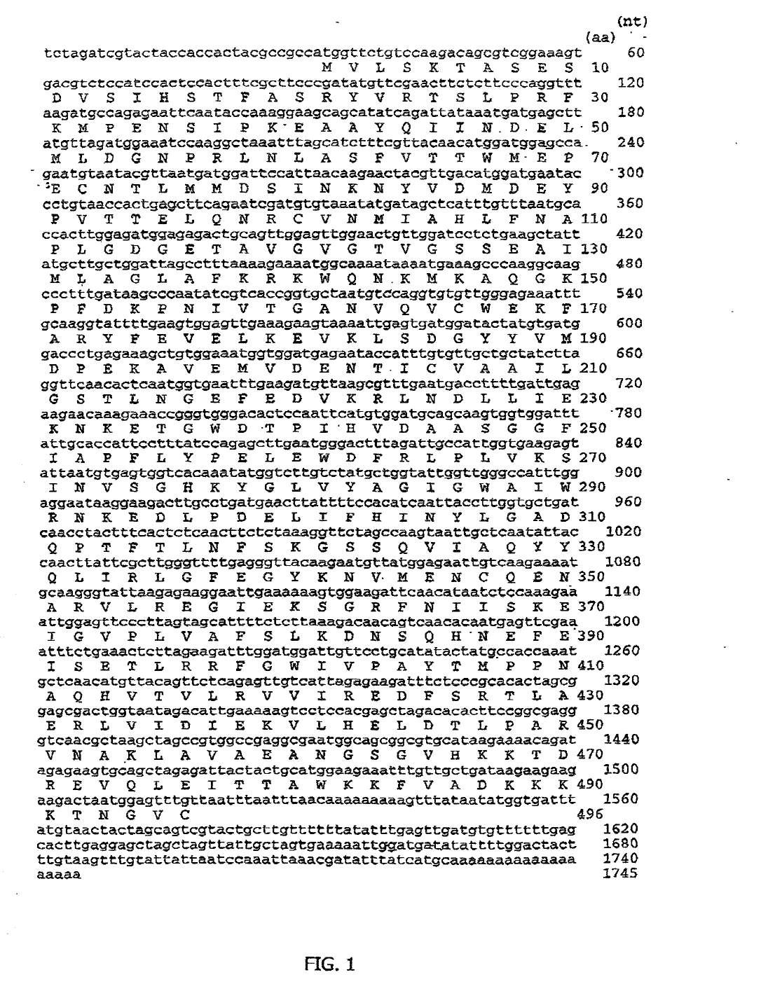

Having herein provided the nucleotide sequence of a gene encoding tobacco GAD (FIG. 1), it will be appreciated that automated techniques of gene synthesis and/or amplification can be performed to generate GAD-encoding DNA. In this case, because of the length of the GAD-encoding DNA, application of automated synthesis may require staged gene construction in which regions of the gene up to about 300 nucleotides in length are synthesized individually and then ligated in correct succession via designed overlaps. Individually synthesized gene regions can then be amplified by PCR.

Once obtained, the GAD-encoding DNA is incorporated for expression into any suitable expression vector, and host plant cells are transfected therewith using conventional procedures. In accordance with the present invention, the GAD-encoding DNA is modified prior to its incorporation into an expression vector to enhance expression of GAD As described in the specific example that follows, GAD DNA is truncated to yield a protein that does not bind calmodulin. In one embodiment, the GAD DNA is truncated to delete the calmodulin-binding domain (CaMBD) thereof. This domain is generally located at the C-terminal end of GAD; however, its exact location varies slightly from plant to plant. For example, the CaMBD in tobacco GAD is encoded within nucleic acid residues 1268-1488, while petunia GAD is encoded by nucleic acids 1410-1485 (Arazi et al. 1995 Plant Physiol 108:551). The CaMBD functions to regulate GAD activity. GABA expression is greatly enhanced on calmodulin-binding to the CaMBD. Accordingly, modification of the GAD-encoding DNA by deletion of the CaMBD removes the autoinhibition of GAD expression and allows for the overexpression of GABA in the plant. The result of this accumulation of GABA in the present transgenic plants is a reduced susceptibility to invertebrate pests, particularly nematode pests.

Techniques of genetic engineering are further applied to prepare a plant cell line, and subsequently a transgenic plant, that incorporates GAD-encoding DNA and is adapted to express GAD in functional form as a homologous or heterologous product. The construction of such cell lines is achieved by introducing into a selected host cell a recombinant DNA construct in which DNA coding for GAD is associated with expression controlling elements that are functional in the selected host to drive expression of GAD-encoding DNA, thus elaborating the desired GAD protein. The particular cell type selected to serve as host can be any of several cell types currently available in the art, including both prokaryotic and eukaryotic cell types. Yeast cells, such as Saccharomyces cerevisiae, bacterial cells such as E. coli and insect cells represent suitable host cells for expression and production of plant GAD.

A variety of gene expression systems have been adapted for use with plant host cells and are now commercially available. Any one of these systems can be selected to drive expression of the GAD-encoding DNA. These systems, available typically in the form of plasmidic vectors, incorporate expression cassettes the functional components of which include DNA constituting expression controlling sequences, which are host-recognized and enable expression of GAD-coding DNA when linked 5′ thereof. GAD-encoding DNA is herein referred to as being incorporated “expressibly” into the system, and incorporated “expressibly” in a cell once successful expression from a cell is achieved. These systems further incorporate DNA sequences which terminate expression when linked 3′ of the GAD-encoding region. Thus, for expression in the selected cell host, there is generated a recombinant DNA expression construct in which the GAD-encoding DNA is linked with expression controlling DNA sequences recognized by the host, and which include a region 5′ of the GAD-encoding DNA to drive expression, and a 3′ region to terminate expression.

Included among the various recombinant DNA expression systems that can be used to achieve plant cell expression of the GAP encoding DNA are those that exploit viral or plant promoters that infect plant cells; examples of such promoters include those that are constitutive e.g. CaMV 35S or the “superpromoter” which is apparently several-fold stronger than the CaMV 35S promoter [Ni et al 1995 Plant J. 7:661-676]). Root-specific constitutive promoters may also be used. Examples include the promoter for the tobacco gene TobRB7, which encodes a membrane protein believed to function as a water channel protein (Yamamoto et al 1991 Plant Cell 3: 371-382; Yamamoto et al 1993 Plant J 4:863); and the promoter for the soybean gene SbPRP1, which encodes a cell wall protein (Hong et al 1987 J Biol Chem 262:8367; Suzuki et al 1993 Plant Mol Biol 21:109). Other useful promoters include those which are specifically induced in the feeding cells of an invertebrate pest thereby ensuring that GABA accumulates in the vicinity of the pest. For example, a promoter which is induced in the feeding cells of a parasitic nematode, i.e. a nematode-induced promoter, such as the Δ0.3TobRB7-5A promoter (Yamamoto et al 1991 Plant Cell 3:371; Opperman et al 1994 Science 263:261) is advantageous. Alternatively, tissue- or organ-specific promoters may be used (e.g. Cho et al. 1996 Plant Molec. Biol. Rep. 13: 255-269), temporal-specific promoters (e.g. Gould 1988 Bioscience 38: 26-33) or environmentally-inducible promoters which may be induced by spraying with an environmentally benign chemical (e.g. Williams et al. 1992 Bio/Technology 10: 540-543; Mett et al. 1996 Transgenic Res. 5: 105-113)

Transgenic plants are then generated from plant cells successfully transformed with the GAD-encoding DNA of interest using well-established techniques such as the Agrobacterium-mediated transformation of leaf disks or cotyledons (Meisner et al. (1997) Plant J. 12:1465), Agrobacterium-mediated vacuum infiltration transformation of the ovule (Ye et al. (1999) Plant J. 19:249) and microprojectile bombardment (Christou et al. (1994) Plant Molecular Biology Manual, 2^{nd} Ed. Dodrecht Kluver Academic, pp. A211). Specific techniques that can be used are described in more detail in the specific example that follows.

Transgenic plants incorporating GAD-encoding DNA truncated such that the GAD expressed therefrom does not bind CaMBD are capable of over-expressing GABA to the extent that they exhibit a significant reduction in susceptibility to invertebrate pests, particularly parasitic nematodes. Examples of nematodes against which the present transgenic plants have reduced susceptibility include, but are not limited to, Meloidogyne spp., such as M. hapla, M incognita, M. exigua, M. indica, M. javanica, M. africana, M. graminis, M. gaminicola and M. arenaria; Heterodera spp., such as H. mexicana; H. avenae, H. glycine, H. orqzae, H. schachtii, H. trifolii, H. carotae, H. cruciferae, and H. goettingiana; Globodera spp., such G. rostochiensis, G. pallida, and G. tabacum; and Pratylenchus spp., such as P. penetrans and P. scribneri. Moreover, there is no restriction with respect to the plant type that may be transformed with truncated GAD-encoding DNA in accordance with the present invention. Any transformable monocot or dicot plant affected by parasitic nematodes, such as those listed above, may be transformed in accordance with the present invention to reduce susceptibility to the nematode.

In addition to a significant reduction in susceptibility to nematodes and insect pests that feed on roots, the transgenic plants of the present invention may also exhibit a reduced susceptibility to insect pests that attack plant shoots. This is due to an increased accumulation of GABA also in the plant's shoots, i.e. stems and leaves, in comparison to wild-type plants. Examples of insects against which the present plants may have reduced susceptibility include insects of the orders Lepidoptera, Orthoptera, Dermaptera, Isoptera, Thysanoptera, Heteroptera, Homoptera, Coleoptera, Hymenoptera and Diptera.

The present transgenic plants overexpress GAD such that GABA content increases to a significant extent. Although, the transgenic plants exhibit increased GABA levels in both the shoot and the root, the most significant accumulation of GABA occurs in the plant roots at levels which reduce susceptibility to invertebrate pests in comparison to wild-type plants, e.g. GABA levels at least 100 nmol g⁻¹ fresh weight in the present plants at rest in comparison to very low levels (insignificant) in wild-type plants. In a preferred embodiment, stress-induced GABA root levels of at least 500 nmol g⁻¹ FW occur in the present transgenic plants, and more preferably, root GABA levels of more than 1000 nmol g⁻¹ FW occur under stress. These levels represent at 3-4 fold increase of GABA production in the present transgenic plants under stress-induced conditions in comparison to wild-type plants also under stress-induced conditions. The term “stress-induced conditions” is meant to refer to stimuli which induce a plant's defense mechanisms. These stimuli include insect walking and feeding (i.e. biotic stress) and temperature shock (i.e. abiotic stress).

Moreover, despite the increased levels of GABA throughout the present transgenic plants, they remain phenotypically normal in comparison to wild-type plants with innate levels of GABA. In this regard, the present plants do not exhibit significant reduced growth or function of any facet, and fertility is also maintained. These features are particularly advantageous with cash crops, such as tobacco, soy bean and cereal crops, where above-ground yield is crucial.

Embodiments of the present invention will be described in more detail in the following specific example which is not to be construed as limiting.

### EXAMPLE 1

### Susceptibility to the Root-Knot Nematode of Transgenic Tobacco Plants Overexpressing Tobacco Glutamate Decarboxylase

Plant Material

For determination of GABA levels, transgene copy number, and bulking of seed tobacco plants (Nicotiana tabacum L cvs. Samsun NN and Delgold) were grown either aseptically or in the Guelph greenhouse in a soil mix containing Sunshine Mix 2 (Sun Gro Horticulture Inc. Bellevue, Wis., USA).

cDNA Library Preparation and Screening

A cDNA library was constructed with reverse transcribed poly(A)⁺ RNA isolated from mature leaves of ‘Samsun NN’ tobacco and cloned, using the ZAP-cDNA synthesis kit, into the Uni-ZAP XR vector (Stratagene, La Jolla, Calif.). Recombinant bacteriophage were packaged in vitro using the Gigapack kit (Stratagene). A 1.35-kb BamHI/EcoRI fragment of a petunia GAD cDNA (Baum et al. 1993 EMBO J. 15:2988-2966) was labeled with [α-³²P]dCTP using the Prime-it II random primer labeling kit (Stratagene), and used to probe 1.1×10⁶ recombinant bacteriophage cDNA clones which were blotted onto Gene Screen Plus hybridization transfer membranes (NEN Life Science Products, Boston, Mass.). Prehybridization and hybridization were performed at 55° C. in an aqueous buffer containing 10% dextran sulphate (Na salt, MW 500,000), 1% SDS, 1 M NaCl, 100, λμg/ml denatured and sonicated salmon sperm DNA. The final wash of the membranes was in 0.2×SSC and 0.1% SDS at 60° C. for 30 min (Sambrook et al. 1989 Molecular Cloning: A Laboratory Manual, Second Edition, Cold Spring Harbour Lab Press). Positive plaques were isolated and subjected to secondary and tertiary screening under the same conditions. cDNA-inserts of the positive plaques from the tertiary screening were excised in vivo according to the Stratagene protocol, and insert-containing clones of Bluescript phagemids in the E. coli SOLR strain were analyzed by DNA restriction enzyme analysis, hybridization analysis using the petunia GAD probe, and DNA sequencing.

Plasmid Construction

One cDNA clone called GAD20.1, appeared to contain a full-length GAD cDNA sequence. A two-nucleotide deletion in codon 18 was detected, causing a frame-shift mutation with a premature stop codon after amino acid 29. This was repaired with a DNA fragment produced using the polymerase chain reaction with primers 5′-GGAGTCCATCATAAGCTTATT-3′ (SEQ ID No: 3) and 5′-CTTCTAGATCGTACTACCACCACTACGCC-3′ (SEQ ID No: 4) and tobacco ‘Samsun NN’ cDNA as a template. This fragment was cloned into the 5′ end of the GAD 20.1 cDNA taking advantage of an EcoRI site between codons 34 to 36. The sequence of the repaired GAD20.1 cDNA, which encodes a predicted 496 amino acid polypeptide, appears in FIG. 1.

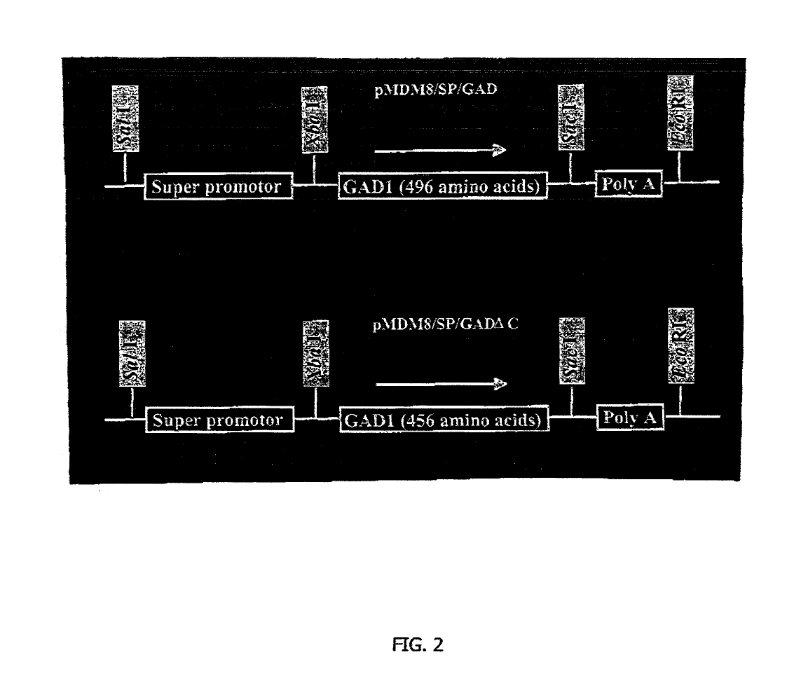

The repaired GAD20.1 cDNA was subcloned downstream of the chimeric octopine synthase/mannopine synthase ‘superpromoter’ between the XbaI and SacI restriction endonuclease sites in pE1068, provided by Dr. Stanton B. Gelvin of Purdue University (Ni et al. 1995 Plant J 7: 661-676). The resulting ‘superpromoter’/GAD20.1 (SPGAD20.1) gene cassette was excised using SalI and SacI restriction endonucleases and cloned into pMDM8, a plant binary transformation vector produced in this laboratory as a derivative of pBIN19 (Frisch et al. 1995 Plant Mol Biol 27: 405-409). pMDM8 differs from pBIN19 by the introduction of two yeast flip recombination target (FRT) sequences (5′-GAAGTTCCTATACTTTCTAGAGAATAGGAACTTC-3′; (SEQ ID No: 5) Lyznik et al. 1996 Nucl. Acid Res. 24:3784-3789) at the PmeI and ClaI restriction sites which flank the neomycin phosphotransferase/kanamycin resistance gene (nptII; Beck et al. 1982 Gene 19:327-336), and the presence of a 260-bp polyadenylation sequence from the nopaline synthase gene (Nos terminator, Depicker et al. 1982 J Mol Appl Genet 1:561-573) between the SacI and EcoRI sites. The T-DNA region of the resultant derivative pMDM8 plasmid bearing the superpromoter/GAD20.1, called pSPGAD20.1, is shown in FIG. 2.

A calmodulin-binding domain (CaMBD) is encoded at the carboxyl-terminal end of the predicted GAD20.1 polypeptide. One CaMBD deletion was prepared from the plasmid shown in FIG. 2. The CaMBD-deletion plasmid, pSPGAD20.1ΔC40, contains a deletion of the 40 carboxyl-terminal amino acids from the GAD20.1 polypeptide, and was made by digesting pSPGAD20.1 with NheI, preparing and then ligating the 9.0 - and 4.7-kb fragments together so as to delete the 235-bp NheI fragment, which contains the GAD20.1 CaMBD. The resulting coding sequence has a serine-tyrosine-cysteine three-amino-acid sequence after amino acid number 456 (alanine), resulting in a predicted polypeptide of 459 amino acids. This constuct is also shown in FIG. 2.

Transgenic Plant Production

Transgenic ‘Delgold’ plants, harboring the T-DNA regions from pSPGAD20.1 and pSPGAD20.1ΔC40 were produced by Agrobacterium-mediated leaf disk transformation (Horsch et al. 1985 Science 227: 229-1231). Selection on 200 mg/l kanamycin sulfate was performed in the presence of 500 mg/l cefotaxime on MS medium with 0.1 mg/l NAA and 1 mg/l BAP. Regenerated shoots were rooted in hormone-free MS medium containing 100 mg/l kanamycin, transferred to soil and grown, after one week under shade cloth, to maturity under greenhouse conditions. Primary transformants were designated T₀ plants.

Derivation of Homozygous Lines, and Determination of GABA Levels

WT plants and primary transformants (62 SPGAD20.1 and 39 SPGAD20.1ΔC40 plants) were grown individually in 9-L pots. After two months of growth, the tips of young leaves (two per plant) at about one-third full expansion, were removed and rapidly frozen in liquid nitrogen. One leaf of each pair was ground in a chilled mortar and pestle with 5 volumes of sulphosalicylic acid (30 mg mL⁻¹). The other leaf of the pair was allowed to thaw for 15 min before being ground. The homogenates were centrifuged in a microfuge, and the supernatants removed and adjusted to pH 7 with 4 N NaOH and stored at −20° C. prior to GABA analysis by reverse-phase high-performance liguid chromatography (Oaks et al. 1986 In Lambers et al. (eds) Fundmental, Ecological and Agricultural Aspects of Nitrogen Metabolism in Higher Plants, Martinus Nijhoff Publ., Dordrecht, Netherlands, Pp 197-202).

In the absence of cold stress, the GABA concentrations in WT controls were not detectable using HPLC methods, whereas those in primary SPGAD20.1 and SPGAD20.1ΔC40 transformants were detectable, with concentrations of 61 and 142 nmol g⁻¹ FW, respectively (Table 1). Note that the plant containing GAD without the CaMBD (i.e. SPGAD20.1ΔC40 ) shows a 2-3 fold increase in GAD in the absence of stress. In the presence of cold stress, GABA concentrations increased in all genotypes with those in the transgenics exceeding at in the WT by 3-4 fold. These data confirm that these cDNAs encode proteins with the hypothesized GAD activities.

TABLE 1
GABA pools in young leaves of greenhouse-grown wild-type and
primary transformants of ‘Delgold’ tobacco plants after abrupt
freezing with liquid nitrogen, followed by a 15-min period at
room temperature. Data represent the mean ± S.E.; the sample
number is given in parentheses. ND indicates not detected.

GABA (nmol g⁻¹ FW)

| Genotype | −Stress | +Stress |
|---|---|---|
| Wild-type | ND (4) | 132.4 ± 4.0 (4) |
| SPGAD20.1 | 61.2 ± 16.9 (5) | 597.1 ± 30.7 (62) |
| SPGAD20.1ΔC40 | 142.3 ± 45.4 (5) | 457.1 ± 25.9 (39) |

The ten plants expressing pSPGAD20.1 with the highest GABA concentrations and the eight plants expressing pSPGAD20.1ΔC40 with the highest GABA concentrations were assayed for GAD20.1 transgene copy number by genomic blot hybridization (Southern 1975 J Mol Biol 98:503-17). DNA samples (2.5 μg/lane) were digested with BclI and separated electrophoretically in a 1% agarose gel using TBE buffer (Sambrook et al. 1989 Molecular Cloning: A Laboratory Manual, Second Edition, Cold Spring Harbour Lab Press). After denaturing the DNA and neutralizaton of the gel, the DNA was capillary-transferred and fixed to Gene Screen Plus membranes NEN Life Sciences) following the manufacturer's instructions. Prehybridization was carried out at 65° C. for 4 h in 10 mL of an aqueous solution containing 10% dextran sulphate (Na salt, MW 500,000), 1% SDS, 1 M NaCl and 100 μg/ml denatured sonicated salmon sperm DNA. Hybridization was performed at 65° C. overnight in the same solution containing a 789-bp GAD20.1 DNA fragment (from a BclI restriction site at codon number 310 to the end of the 3′ untranslated region) as a probe labeled with [α-³²P]dCTP to a specific activity of >10⁹ cpm/μg using the random priming method. The filters were washed at room temperature in 2×SSC, 0.1% SDS, and then for 30 min at 55° C. in 1×SSC, 0.1% SDS, after which they were exposed to X-OMAT film (Kodak, Rochester, N.Y.) for 48 h at −80° C. using intensifying screens. Plants with one or two transgene copies were chosen for further analysis and derivation of homozygous lines. These plants were allowed to set seed and 10 to 20 progeny from each (T₁ generation) were grown and also allowed to set seed (T₂ generation). DNA was purified from the T₁ plants and analyzed by genomic blot hybridization as above. Homozygotes for each transgene band were selected by judging hybridization intensities on the produced autoradiograms; plants with double intensity transgene bands were selected as homozygotes, whereas single intensity hemizygotes and null homozygotes were discarded. Seeds of the wild-type and ten homozygous lines of SPGAD20.1 and SPGAD20.1 ΔC40 were grown on filter paper soaked with 1 mM calcium sulphate in petri dishes in the dark at room temperature. After two weeks, the shoots were rapidly removed by scraping them into liquid nitrogen. The samples were then ground in a chilled mortar and pestle in five volumes of sulphosalicylic acid (30 mg mL⁻¹, and the GABA concentrations determined as described above. With the exception of SP20.1 ΔC40/6.12, all lines had significantly higher GABA levels (Table 2). Alternatively, seeds of wild type and the homozygous lines of SPGAD20.1 and SPGAD20.1ΔC40 were individually germinated in magenta boxes on hormone-free MS agar medium containing 2% (w/v) sucrose, and grown under the culture room conditions described above. After 12 weeks, each shoot was rapidly removed with scissors and placed in liquid nitrogen. Then the roots were slowly removed from the medium using tweezers where necessary and frozen in liquid nitrogen. The samples were allowed to thaw for 10 min, and then were ground in a chilled mortar and pestle with 5 volumes of sulphosalicylic acid (30 mg mL⁻¹). The GABA contents of the homogenates were determined as described above.

In seven of the eleven homozygous lines measured, shoot GABA concentrations were significantly higher than the corresponding WT plants (Table 3), whereas six of the transgenic lines had higher GABA root levels than the WT. In transgenic lines containing GAD20.1 without the CaMBD, GABA levels were elevated up to about 3500 nmol g⁻¹ FW, a value 24 times that in wild-type plants.

TABLE 2
GABA pools in shoots of 14-day-old wild-type ‘Delgold’ tobacco
seedlings and homozygous transgenic seedlings (‘Delgold’)
overexpressing, under the control of the ‘superpromoter’ (SP), a
full-length tobacco GAD (GAD20.1) or a truncated tobacco GAD
lacking the carboxyl terminal 40 amino acids (GAD20.1ΔC40).
The seedlings were grown on filter paper in the dark and supplied
with 1 mM calcium sulphate only. Values represent the mean ±
S.E. of three replicate petri dishes containing approximately
0.1 g of seed.

| Genotype | GABA pool (nmol g⁻¹ FW) |
|---|---|
| Wild-type | 6.6 ± 0.5 |
| SPGAD20.1/8.11 | 16.0 ± 2.3 |
| SPGAD20.1/8.17 | 12.0 ± 1.8 |
| SPGAD20.1/10.4 | 17.5 ± 1.2 |
| SPGAD20.1/56.10 | 9.7 ± 0.9 |
| SPGAD20.1/56.17 | 9.8 ± 0.2 |
| SPGAD20.1/56.19 | 17.8 ± 0.9 |
| SPGAD20.1/82.6 | 14.1 ± 0.9 |
| SPGAD20.1ΔC40/6.12 | 8.5 ± 0.8 |
| SPGAD20.1ΔC40/22.1 | 17.5 ± 1.6 |
| SPGAD20.1ΔC40/22.5 | 16.2 ± 0.2 |

TABLE 3
GABA pools in shoots and roots of tissue culture-grown wild-type
‘Delgold’ tobacco plants and homozygous transgenic plants
(‘Delgold’) overexpressing, under the control of the ‘superpromoter’
(SP) a full-length tobacco GAD (GAD20.1) or a truncated tobacco
GAD lacking the carboxyl terminal 40 amino acids (GAD20.1ΔC40).
Plant parts were harvested after 12 weeks, and rapidly frozen in liquid
nitrogen, followed by a 10-min period at room temperature. Data
represent the mean ± S.E. of 5-13 plants. The transgene number was
determined by Southern analysis.

| | | T-DNA | | |
|---|---|---|---|---|
| | | insertions | GABA pool | |
| | | Designa- | (nmol g⁻¹ FW) | |
| Genotype | # Bands | tion | Shoot | Root |
| Wild-type | | | 198 ± 20 | 139 ± 9 |
| SPGAD20.1/8.11 | 1 | 2 | 377 ± 27 | 495 ± 112 |
| SPGAD20.1/8.17 | 1 | 1 | 334 ± 17 | 302 ± 52 |
| SPGAD20.1/10.4 | 1 | 1 | 744 ± 100 | 186 ± 40 |
| SPGAD20.1/56.10 | 1 | 2 | 515 ± 84 | 220 ± 22 |
| SPGAD20.1/56.17 | 1 | 2 | 475 ± 65 | 160 ± 23 |
| SPGAD20.1/56.19 | 2 | 1 & 2 | 226 ± 17 | 263 ± 36 |
| SPGAD20.1/82.1 | 1 | 1 | 294 ± 24 | 165 ± 21 |
| SPGAD20.1/82.6 | 1 | 1 | 606 ± 58 | 235 ± 56 |
| SPGAD20.1ΔC40/6.12 | 1 | 1 | 128 ± 9 | 240 ± 70 |
| SPGAD20.1ΔC40/22.1 | 1 | 2 | 283 ± 43 | 2007 ± 657 |
| SPGAD20.1ΔC40/22.5 | 1 | 1 | 280 ± 31 | 3532 ± 290 |

Nematode Resistance Bioassay

For each bioassay, seeds of a homozygous transgenic line and wild-type ‘Delgold’ tobacco were sown in seedling trays filled with a Fox sandy loam (pH 6.5), and grown to the 3-leaf stage in a controlled environment chamber (a 16-h photoperiod with a day/night temperature of 23/18° C., a combination of inflorescence and incadescent lamps providing a photosynthetic photon flux density of 250 μmol m⁻² s⁻¹ at the top of the tray, 40-65% relative humidity).

Eight 0.6-cm holes were drilled into the bottom of a Rubbermaid™ storage bin (45.7×35.6×30.5 cm) for drainage, then the bin was filled with 29 L of sand containing Meloidogyne hapla. Eight Lycopersicon esculentum L. cv. Bonnie Best seeds were sown into the bin, and grown under the conditions described above for approximately six months, with biweekly detopping of the main stem to the uppermost branch. The tomato plants were removed, and the soil in the bin thoroughly mixed with a shovel. Two troughs were dug in the soil, each about 10 cm from the side of the bin. Two rootrainers (Model Hillson #170-4, Spencer-Lemarie Rootainers, Edmonton, AB), each containing four cells of 170 mL volume, were inserted into each trough. WT and transgenic tobacco plants as described above, were placed in alternate cells of each rootrainer, together with nematode-containing soil. The roots within the rootainer were inoculated, using a syringe, with an additional 2000 eggs and/or J2 juveniles that were freshly collected from infested plants using a sieving method as described elsewhere (Barker 1985 In Barker et al., eds, An Advanced Treatise On Meloidogyne, Vol 2 Methodoloy, North Carolina State University Graphics. Pp 19). The plants were supplied with 3 L of tap water every second day, and once weekly with liquid fertilizer as described above. Two weeks after transplanting sufficient plants were culled so that four healthy plants of each genotype remained. Six weeks after transplanting, the plants were detopped to the sixth node. Nine weeks after transplanting the number of egg masses present on the roots within the cell only, was determined by microscopic examination.

In this experiment, the root phenotype was examined closely and the results are tabulated below in Table 4. In five of the seven SPGAD20.1 screened, there were significantly less reproductive female nematodes on the roots. Of these, three had a normal root weights (8.17, 56.17, 56.19), and one-half to one-third the number of reproductive females found on wild-type plants on both a root and fresh weight basis. Of the two SPGAD20.1ΔC40 lines screened, the root fresh weight was not significantly different from that of wild-type plants, but there was less than 10% the number of reproductive female nematodes on the roots of these plants in comparison to wild-type plants.

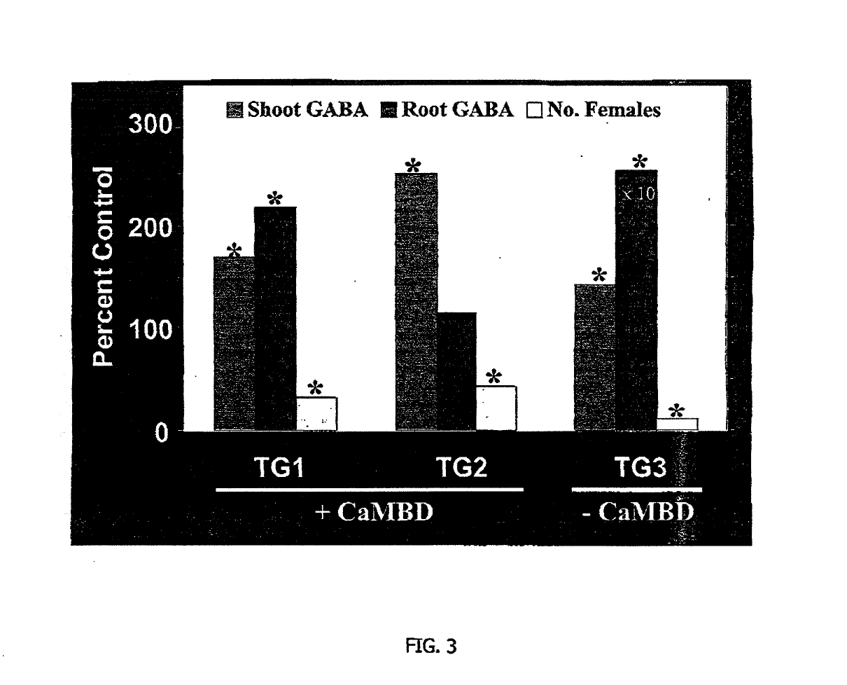

These results are graphically illustrated in FIG. 3 which shows the percentage increase in GABA accumulation during stress, as well the susceptibility to the root knot nematode in 12-week-old tobacco plants. Data are calculated from Tables 3 and 4, and are restricted to transgenic plants with root weight that is not significantly different from the wild-type controls. TG1, TG2 and TG3 represent SPGAD20.1/8.17, SPGAD20.1/56.17, SPGAD20.1ΔC40/22.5, respectively. Note that with SPGAD20.1ΔC40/22.5, the percentage for root GABA levels is multiplied by 10 as indicated on the bar.

The results show that plants with higher GABA-synthesizing capacity, such as the present transgenic plants, are less susceptible to nematodes, and the level of resistance is correlated with GABA levels.

All references referred to herein are incorporated by reference.

TABLE 4
Production of reproductive females in growth-chamber grown wild-
type and transgenic ‘Delgold’ tobacco plants 9-12 weeks after
inoculation. Data represent the mean ± S.E. and should only be
compared between paired wild-type and transgenic plants. * and **
indicate significant difference between wild-type and transgenic
plants at the 95 and 90% confidence limits, respectively, as determined
by a Kruskal-Wallis one way ANOVA and comparison of mean ranks.
Root fresh weight was not analyzed statistically. Line
SPGADΔC40/22.5 was tested twice.

| | | Root FW (g) | Nematode Number | |
|---|---|---|---|---|
| Experiment/Genotypes | | number/root | number/g FW | |
| 1. | Wild-type | 11.8 ± 1.2 | 43 ± 9 | 3.7 ± 0.7 |
| | SPGAD20.1/8.11 | 3.1 ± 1.0 | 16 ± 6** | 5.3 ± 0.5 |
| 2. | Wild-type | 9.7 ± 1.8 | 57 ± 6 | 6.4 ± 1.0 |
| | SPGAD20.1/8.17 | 9.5 ± 1.0 | 18 ± 2* | 1.9 ± 0.2* |
| 3. | Wild-type | 9.7 ± 1.8 | 33 ± 21 | 2.6 ± 1.0 |
| | SPGAD20.1/10.4 | 2.5 ± 0.7 | 0 ± 0* | 0 ± 0* |
| 4. | Wild-type | 8.6 ± 4.4 | 44 ± 17 | 9.8 ± 4.8 |
| | SPGAD20.1/56.10 | 7.3 ± 2.1 | 23 ± 10 | 3.0 ± 0.6 |
| 5. | Wild-type | 6.2 ± 0.8 | 63 ± 16 | 9.6 ± 1.6 |
| | SPGAD20.1/56.17 | 6.8 ± 1.0 | 27 ± 6* | 4.7 ± 1.6** |
| 6. | Wild-type | 10.4 ± 2.2 | 133 ± 14 | 13.9 ± 2.1 |
| | SPGAD20.1/56.19 | 13.1 ± 3.2 | 52 ± 17* | 5.0 ± 1.9* |
| 7. | Wild-type | 8.3 ± 2.1 | 80 ± 21 | 12.0 ± 3.4 |
| | SPGAD20.1/82.6 | 4.5 ± 0.4 | 96 ± 17 | 21.0 ± 2.7 |
| 8. | Wild-type | 10.0 ± 3.2 | 107 ± 19 | 12.0 ± 1.7 |
| | SPGADΔC40/6.12 | 6.7 ± 1.6 | 94 ± 28 | 17.5 ± 7.5 |
| 9. | Wild-type | 8.3 ± 2.1 | 22.1 ± 4.2 | 4.0 ± 1.3 |
| | SPGAD20.1ΔC40/22.1 | 5.6 ± 1.1 | 7.2 ± 1.4* | 1.5 ± 0.4** |
| 10. | Wild-type | 9.0 ± 2.3 | 21 ± 10 | 2.7 ± 1.1 |
| | SPGAD20.1ΔC40/22.5 | 5.6 ± 0.9 | 2.0 ± 0.7* | 0.5 ± 0.2** |
| 11. | Wild-type | 4.5 ± 1.4 | 15 ± 5 | 3.2 ± 1.0 |
| | SPGAD20.1ΔC40/22.5 | 2.7 ± 0.6 | 0.5 ± 0.5* | 0.2 ± 0.2* |

We claim:

1. A phenotypically normal transgenic plant having reduced susceptibility to invertebrate pests, wherein the plant is transformed with a nucleic acid which expressibly encodes a glutamate decarboxylase (GAD).

2. A transgenic plant as defined in claim 1, wherein the nucleic acid comprises a GAD-encoding gene which lacks the calmodulin-binding domain.

3. A transgenic plant as defined in claim 1, wherein the GAD-encoding gene is under the control of a root-specific promoter.

4. A transgenic plant as defined in claim 2, wherein the GAD-encoding gene encodes GAD having the amino acid sequence of residues 1-456 as set out in SEQ ID No: 2.

5. A transgenic plant as defined in claim 2, wherein the GAD-encoding gene has a nucleic acid sequence of residues 31-1398 as set out in SEQ ID No: 1.

6. A transgenic plant as defined in claim 1, wherein the GAD-encoding gene is under the control of a nematode-induced promoter.

7. A transgenic plant as defined in claim 1, wherein the invertebrate pests are root-feeding pests.

8. A transgenic plant as defined in claim 7, wherein the root-feeding pests are nematodes.

9. A transgenic plant as defined in claim 1, having a GABA level of at least 100 nmol g⁻¹ FW.

10. A transgenic plant as defined in claim 1, having a stress-induced root GABA level of at least 500 nmol g⁻¹ FW.

11. A transgenic plant as defined in claim 10, having a stress-induced root GABA level of at least 1000 nmol g⁻¹ FW.

12. A method of producing a phenotypically normal transgenic plant having enhanced resistance to invertebrate pests, comprising:
1) transforming a recipient plant cell with a recombinant nucleic acid expressibly encoding glutamate decarboxylase;
2) generating a plant from the transformed plant cell; and
3) selecting for a phenotypically normal transformed plant having a GABA level of at least 100 g/nmol fresh weight.

13. A method as defined in claim 12, wherein the nucleic acid comprises a GAD-encoding gene which lacks the calmodulin-binding domain.

14. A method as defined in claim 13, wherein the plant selected has a stress-induced root GABA level that is at least 500 nmol g⁻¹ FW higher than wild-type levels.

15. A method as defined in claim 13, wherein the GAD-encoding gene encodes GAD having the amino acid sequence of residues 1-456 as set out in SEQ ID No: 2.

16. A method as defined in claim 12, wherein the invertebrate pests are root-feeding pests.

17. A method as defined in claim 16, wherein the root-feeding pests are nematodes.

18. A method as defined in claim 10, wherein the GAD-encoding gene is under the control of a root-specific promoter.

19. A method as defined in claim 12, wherein the plant selected has a stress-induced root GABA level of at least 500 g/nmol fresh weight.

20. A method as defined in claim 19, wherein the plant has a stress-induced root GABA level of at least 1000 g/nmol fresh weight.
